(19) United States
(12) Patent Application Publication
(10) Pub. No.: US 2008/0176907 A1
(43) Pub. Date: Jul. 24, 2008

(54) NOS Inhibitors For Treatment Of Motor Deficit Disorders

(75) Inventors: Richard B. Silverman, Northbrook, IL (US); Haitao Ji, Evanston, IL (US); Sidhartha Tan, Wilmette, IL (US); Matthew Derrick, Winnetka, IL (US)

Correspondence Address: Casimir Jones, S.C., 440 Science Drive, Suite 203, Madison, WI, 53711, US

(73) Assignee: Northwestern University, Evanston, IL (US)

(21) Appl. No.: 11/857,626

(22) Filed: Sep. 19, 2007

Related U.S. Application Data
(60) Provisional application No. 60/845,679, filed on Sep. 19, 2006.

(51) Int. Cl. A61K 31/4439; A61P 25/00

(52) U.S. Cl. 514/343

(57) ABSTRACT

The present invention relates to preventive therapies and treatments of motor deficit disorders. In particular, the present invention relates to compositions and methods for preventative therapy and treatment of motor deficit disorders, such as cerebral palsy, in fetuses and newborn infants using inhibitors to neuronal nitric oxide synthase (nNOS).

The present application claims priority to U.S. Provisional Application No. 60/845,679, filed Sep. 19, 2006.

This invention was made with government support under grant numbers GM49725 and NS43285, awarded by the National Institutes of Health. The government has certain rights in the invention.

### FIELD OF THE INVENTION

The present invention relates to preventive therapies and treatments of motor deficit disorders. In particular, the present invention relates to compositions and methods for preventative therapy and treatment of motor deficit disorders, such as cerebral palsy, in fetuses and newborn infants using inhibitors to neuronal nitric oxide synthase (nNOS).

### BACKGROUND OF THE INVENTION

Cerebral palsy is a general term used to encompass a group of motor disorders and conditions that affect control of movement and posture. Because of damage to one or more parts of the brain that control movement, an affected child cannot move his or her muscles normally. While symptoms range from mild to severe, the condition generally does not worsen with age. Unfortunately, many children with some form of cerebral palsy have other conditions that require further treatment, such as mental retardation, learning disabilities, seizures, vision, hearing and speech problems. Most will require some form of specialized care for the whole of their lives. About two to three children in 1,000 over the age of three have some form of cerebral palsy, such that around 500,000 children and adults of all ages in this country are afflicted.

Even though in many cases the cause of cerebral palsy is not known, some known causes of cerebral palsy include; infections during pregnancy that can cause brain damage or damage to the placental membranes (e.g., German measles, viral infections, parasitic infections), insufficient oxygen reaching the fetus (e.g., placental insufficiency), premature births (e.g., tiny babies suffering from brain damage due to bleeding, oxygen deprivation, ischemia), asphyxia during labor and delivery, blood incompatibilities between the mother and the fetus (e.g., Rh disease), severe jaundice that leads to brain damage, other birth defects (e.g., babies with other physical or genetic birth defects are at increased risk for developing cerebral palsy) and acquired cerebral palsy after birth due to a brain injury (e.g., infections such as meningitis or head injuries).

There is no cure for cerebral palsy and there is no known preventative treatment for treating a pregnant mother for potential oxygen deprivation to the fetus or diminishing the effects of asphyxia during birth. As such, what are needed are compositions and methods for preventative treatments and therapies for motor deficit disorders for pregnant women who are at high risk of delivering a child that could suffer from cerebral palsy.

What are also needed are tools to help researchers define more specifically the causes of these motor disorders, such that more effective methods of preventative treatments and therapies can be designed and administered.

### SUMMARY OF THE INVENTION

The present invention relates to preventive therapies and treatments of motor deficit disorders. In particular, the present invention relates to compositions and methods for preventative therapy and treatment of motor deficit disorders, such as cerebral palsy, in fetuses and newborn infants using inhibitors to neuronal nitric oxide synthase (nNOS). Nitric oxide production results in formation of reactive nitrogen species that have been implicated in cell death and apoptosis cascades following hypoxia-reoxygenation. Nitric oxide derived reactive species increase in fetal brain following in vivo hypoxia-reoxygenation in rabbits (Tan et al., 1999, J. Neuropathol. Exp. Neurol. 58:972-981). Pre-term hypoxia-ischemia in pregnant rabbits results in cerebral palsy phenotypes of motor, sensory and reflex deficits in newborn rabbits (Tan et al., 2001, Ped. Acad. Soc. Ann. Mtg. Abstract #2485).

The present application discloses compositions and methods for preventative treatments for pregnant subjects with a risk of delivering premature babies, or with a risk of delivering babies with birth defects (congenital or otherwise) caused by, for example, prenatal acute placental insufficiency that may inhibit or decrease the amount of oxygen reaching the fetus due to ischemia or other factors. The methods and compositions herein disclosed are also applicable for treating pregnant subjects whose newborn may experience asphyxia during birth.

In one embodiment, the present invention comprises a method of preventing a motor deficit disorder in a fetus or newborn infant by administering to a pregnant subject having a risk of delivering a fetus or newborn infant with a motor deficit disorder, or a fetus or newborn, a neuronal nitric oxide synthase inhibitor. In some embodiments, said nitric oxide synthase inhibitor comprises cis-N¹-[4′-6″-Amino-4″-methyl-pyridin-2″-ylmethyl)-pyrrolidin-3′-yl]-N²-(4′-chloro-benzyl)-ethane-1,2-diamine, cis-N¹-[4′-6″-Amino-4″-methyl-pyridin-2″-ylmethyl)-pyrrolidin-3′-yl]-N²-phenylethyl-ethane-1,2-diamine, and/or cis-N¹-[4′-6″-Amino-4″-methyl-pyridin-2″-ylmethyl)-pyrrolidin-3′-yl]-N²-[2′-(3″fluoro-phenyl)-ethyl]-ethane-1,2-diamine. Other exemplary compounds and formulations are found in U.S. Pat. Publication 2005/0159363, herein incorporated by reference in its entirety.

In some embodiments, the present invention comprises compositions for preventing motor deficit disorders in fetuses and newborn children. In some embodiments, the compositions comprise Formula I, Formula II, and/or Formula III.

In some embodiments, the present invention provides for the use of a neuronal nitric oxide synthase inhibitor for the manufacture of a medicament for use in the treatment of a fetus or newborn, by administration to a pregnant subject, of a motor deficit disorder

The present invention also provides methods using pharmaceutical composition comprising a composition of this invention in conjunction with a physiologically or otherwise suitable formulation. In a some embodiments, the present invention includes one or more NOS inhibitors as set forth above formulated into compositions together with one or more non-toxic physiologically tolerable or acceptable diluents, carriers, adjuvants or vehicles that are collectively referred to herein as diluents, for parenteral injection, for oral administration in solid or liquid form, for rectal or topical administration, or the like. The resulting compositions can be, in conjunction with the various methods described herein, administered to humans and animals either orally, rectally, parenterally, intracisternally, intravaginally, intraperitoneally, locally, or as a buccal or nasal spray.

Compositions suitable for parenteral administration can comprise physiologically acceptable sterile aqueous or nonaqueous solutions, dispersions, suspensions or emulsions and sterile powders for reconstitution into such sterile solutions or dispersions. Examples of suitable diluents include water, ethanol, polyols, suitable mixtures thereof, vegetable oils and injectable organic esters such as ethyl oleate. Proper fluidity can be maintained, for example, by the use of a coating such a lecithin, by the maintenance of the required particle size in the case of dispersions and by the use of surfactants.

Compositions can also contain adjuvants such as preserving, wetting, emulsifying, and dispensing agents. Prevention of the action of microorganisms can be insured by various antibacterial and antifungal agents, for example, parabens, chlorobutanol, phenol, sorbic acid, and the like. It may also be desirable to include isotonic agents, for example, sugars, sodium chloride and the like. Prolonged absorption of the injectable pharmaceutical form can be brought about by the use of agents delaying absorption, for example, aluminum monostearate and gelatin. Besides such inert diluents, the composition can also include sweetening, flavoring and perfuming agents. Suspensions, in addition to the active compounds, may contain suspending agents, as for example, ethoxylated isostearyl alcohols, polyoxyethylene sorbitol and sorbitan esters, microcrystalline cellulose, aluminum metahydroxide, bentonit, agar-agar, and tragacanth, or mixtures of these substances, and the like.

### DEFINITIONS

As used herein, the term “subject” refers to any animal (e.g., a mammal), including, but not limited to, humans, non-human primates, rodents, and the like, which is to be the recipient of a particular treatment.

As used herein, the term “cerebral palsy” refers to a class of motor deficit disorders that can occur, for example, due to oxygen deprivation to the fetus or newborn infant during birth. For example, as described herein placental insufficiency can cause a decrease or inhibition of oxygen flow to the fetus for short or long term periods of time. Asphyxia during the birthing process can also decrease oxygen to the infant and be a cause of cerebral palsy.

### FIGURES

FIG. 1 shows structural diagrams of compounds HJ619, JI6 and JI8.

FIG. 2 shows binding of exemplary compounds of the present invention to binding sites on target molecules. FIG. 2A shows one of the cis enantiomers, the (3′S,4′S)-isomer of an exemplary compound 4, bound to an active site. The binding conformation in the crystal structure is very similar to that predicted in the fragment-based de novo design shown in FIG. 2B and is very similar to the binding modes of 1-3 shown in FIG. 2C.

FIG. 3 shows data presenting resulting death and neurobehavioral abnormalities in newborn kits upon treatment with exemplary compounds of the invention.

FIG. 4 illustrates various schemes (A, B, and C) for synthesizing exemplary compounds (A) 4 and (B) 5 of the present invention.

FIG. 5 shows the residues in the active site of NOS that are within 6 Å of bound substrate L-arginine. The amino acid sequences of NOS were retrieved from the PIR protein sequence database. The sequences are human nNOS (entry P29475), rat nNOS (entry P29476), human eNOS (entry P29474), bovine eNOS (entry P29473), human iNOS (entry P35228), and murine iNOS (entry P29477).

FIG. 6 shows eighteen out of 30 residues/cofactors pointing into the active site of nNOS are polar and/or charged. The acidic residues and cofactor are: D495, E592, D597, D600, D709, and heme propionates. The basic residues are: R414, R481, R596, and R603. The polar residues and cofactor are: S477, Q478, Y562, N569, Y588, S602, Y706, and H₄B. The nonpolar residues are: M336, L337, A497, W561, P565, V567, M570, F584, G586, W678, W711, and W306. B. Clusters of acidic residues in the active site of nNOS. For instance: E592, D597 and one heme propionate group and two low pKₐ polar side chains (Y562 and Y588) form a local acidic environment; Y706 and the other heme propionate group form another local acidic environment.

### DETAILED DESCRIPTION OF THE INVENTION

Nitric oxide synthase (NOS) comprises a family of enzymes, including neuronal (nNOS), inducible (iNOS), and endothelial (eNOS) isozymes, which catalyzes the oxidation of L-arginine to L-citrulline and nitric oxide (NO). Neuronal NOS knockout neonatal animals are protected from neonatal hypoxia-ischemia (HI)-induced brain damage (Ferriero et al., 1996, Neurobiol. Dis. 3:64-71); inhibition of nNOS prior to HI confers resistance to HI-induced brain damage (Ferriero et al., 1995, Pediatr. Res. 38:912-918). HI-induced brain damage in iNOS knock-out animals also is reduced (Iadecola et al., 1997, J. Neurosci. 17:9157-9164). However, the expression of nNOS, but not iNOS, is increased dramatically after cerebral HI in the newborn rat (vand den Tweel et al., 2005, J. Neuroimmunol. 167:64-71). By contrast, animals lacking the eNOS gene have enlarged cerebral infarcts after HI (Huang et al., 1996, J. Cereb. Blood Flow Metab. 16:981-987). Prior administration of slightly selective nNOS inhibitors, such as 2-iminobiotin (van den Tweel et al., 2005, J. Cereb. Blood Flow Metab. 25:67-74) and 7-nitroindazole (Ishida et al., 2001, Brain Dev. 23:349-354) reduces brain damage in neonatal and postnatal models of unilateral carotid ligation and hypoxia. However, treatment with potent nNOS inhibitors that also inhibit eNOS causes sharp increases in blood pressure (Zicha et al., 2003, Clin. Sci. 105:483-489).

In developing embodiments of the present invention, a more clinically relevant model of acute placental insufficiency and global fetal hypoxia-ischemia that results in motor deficits resembling cerebral palsy in humans (Derrick et al., 2004, J. Neurosci. 24:24-34; Tan et al., 2005, J. Child Neurol. 20:972-979) was utilized for investigating cellular and biochemical mechanisms resulting from NO pathways, wherein selective inhibition of NOS was evaluated. Some embodiments of the present invention provide isoform selective-compounds to control the NO overproduction by nNOS while leaving the macrophage function of iNOS and the vasoprotective function of eNOS undisturbed (Erdal et al., 2005, Curr. Top. Med. Chem. 5:603-604; Tafi et al., 2006, Curr, Med. Chem. 13:1929-1946), thereby providing methods, for example, for the safe administration of compounds to mothers to treat fetal brain hypoxia-ischemia.

Despite its initial promise, high-throughput screening (HTS) of libraries of drug-like compounds has not delivered a major breakthrough in the efficiency of drug discovery (Lipinski and Hopkins, 2004, Nature 432:855-861). Fragment-based screening for lead discovery that has been proposed most recently allows for a more effective exploration of chemical diversity space (Erlanson, 2006, Curr. Opin. Biotechnol. 17:643-653; Hajduk and Greer, 2007, Nat. Rev. Drug Discov. 6:211-219) (typically the chemical space of 10⁹ molecules versus 106-10⁷ molecules of HTS). However, it is still a small group of the total chemical diversity space (10⁶⁰ drug-like molecules for 30 non-hydrogen atoms) (Dobson, 2004, Nature 432:824-828). Current fragment-based screening approaches only identify and characterize fragment binding sites on the target protein, the so-called “hot spots” on enzyme surfaces or in the active site that are major contributors to the ligand binding free energy (Vajda nd Huarnieri, 2006, Curr. Opin. Drug Discov. Devel. 9:354-362). Unfortunately, many binding sites that are responsible for the isozyme specificity and/or selectivity are not included in the “hot spots”. On the other hand, conventional de novo design programs have two challenges (Schneider and Fechner, 2005, Nat. Rev. Drug Discov. 4:649-663): binding affinity predictions between receptor and new ligands are poor (Leach et al., 2006, J. Med. Chem. 49:5851-5855), and the synthetic accessibility of the newly designed molecules may be limited. It is rare for conventional de novo computer programs to generate initial novel lead structures with nanomolar activity. Recently, graph framework-based approaches, such as scaffold hopping, have been proposed as new methodologies in the field of de novo ligand design (Barker et al., 2006, J. Chem. Inf. Model. 46:503-511). However, the skeleton of the newly designed molecule is confined to the basic architecture of the template structure.

Some embodiments of the present invention provide new methodologies, called fragment hopping, representing a pharmacophore-driven strategy for fragment-based de novo design. The core of this approach is the determination of the minimal pharmacophoric elements; from these elements, for example, new fragments can be generated and linked. Fragment hopping explores a wider chemical diversity space compared with fragment-based screening and can identify and utilize, for example, not only the “hot spots” for fragment binding, but also regions for ligand selectivity. Compared with conventional de novo design, the binding affinity between receptor and ligands are mapped by minimal pharmacophoric elements, and the ligand synthetic accessibility can be reached, for example, with concomitant side chain libraries and bioisostere swapping. In some embodiments, the present invention provides methodologies for drug design, for example, for use as inhibitors, agonists and/or antagonists useful in treating diseases and disorders.

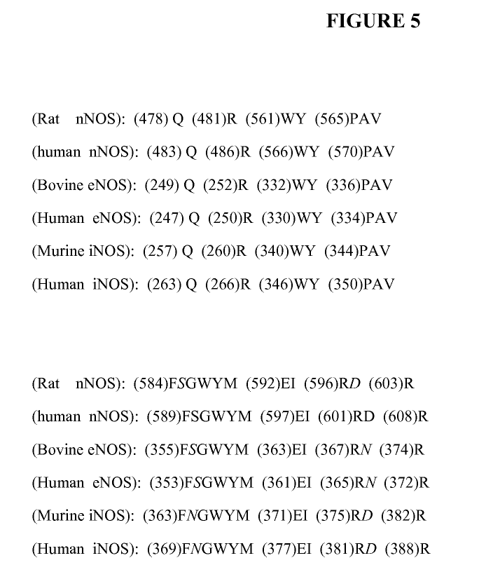

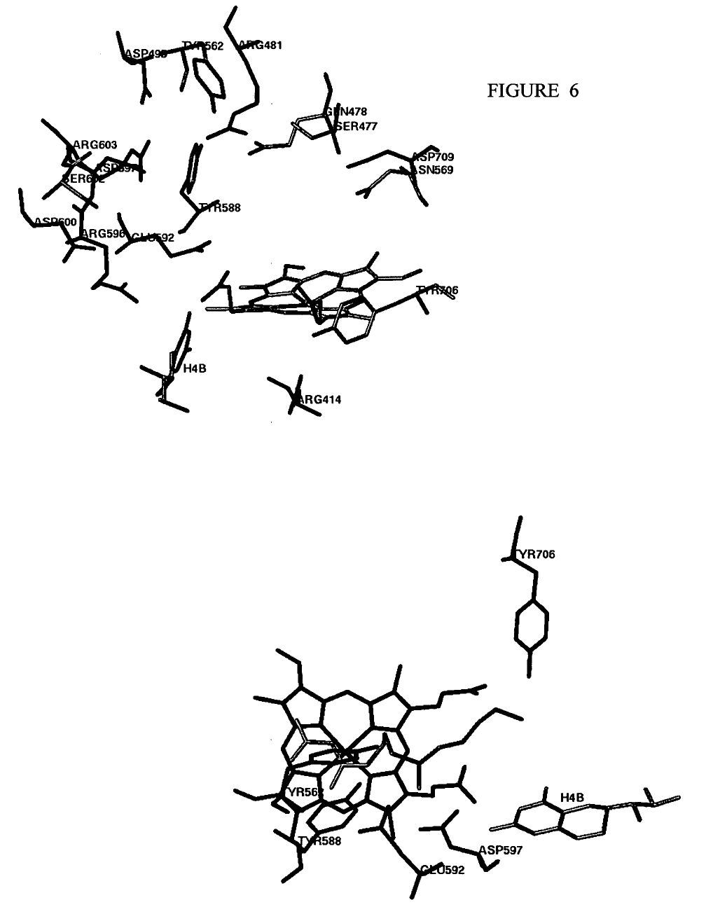

The crystal structures of the oxygenase domains for all three isoforms of NOS are now known Li and Poulos, 2005, J. Inorg. Biochem. 99:293-305), which opens the way for structure-based inhibitor design. However, two key challenges exist in NOS inhibitor design: (1) Selectivity: the active sites of NOS isozymes are highly conserved (FIG. 5). Sixteen out of 18 residues within 6 Å of the substrate in the active site are identical, and the side chain of one of the dissimilar amino acids of nNOS, namely S585, points out of the substrate-binding site, which means that, within that space, only one residue is different between nNOS and eNOS; (2) Permeability: eighteen of 30 residues/cofactor side chains that point into the active site of NOS are polar or charged, and clusters of acidic residues/cofactor side chains, especially residues E592 and D597 (the numbering of residues is based on rat nNOS), the heme propionate groups, and two low pKₐ polar side chains (residues Y562 and Y588), form a strongly acidic local environment (FIG. 6). This environment requires the inhibitor to contain positively charged hydrogen bond donors, such as the primary amine, which creates a problem for the design of a cell-permeable exogenous inhibitor. Multiple numbers of positively-charged hydrogen bond donors are unfavorable for diffusion through biomembranes, such as the blood brain barrier (BBB) (Norinder and Haeberlein, 2002, Adv. Drug Deliv. Rev. 54:291-313).

As shown in Table 1, several synthetic dipeptide amides and peptidomimetics built on an L-N^{ω}-nitroarginine (L-NNA) scaffold (1-3) were discovered and shown to be highly dual-selective inhibitors of nNOS over both eNOS and iNOS (Hah et al., 2003, J. Med. Chem. 46:1661-1669; Gomez-Vidal et al., 2004, J. Med. Chem. 47:703-710). It was previously found that a single-residue difference in the active site, rat nNOS D597 versus bovine eNOS N368, accounts for a majority of the selectivity of nNOS over eNOS by these compounds (Flinspach et al., 2004, Nat. Struct. Mol. Biol. 11:54-49; Ji et al., 2003, J. Med. Chem. 46:5700-5711). This high selectivity is determined by the binding preference of the α-amino groups of 1-3 to a more acidic environment in nNOS. In developing embodiments of the present invention, the compounds as described herein were utilized in a pharmacophore-driven strategy for fragment-based de novo design for identifying non-peptide small molecule inhibitors able to utilize the minute structural differences among the NOS isoforms for treatment of diseases as disorders, for example for the prevention and/or treatment of cerebral palsy. However, it is contemplated that the methods described herein are equally applicable to other isozyme systems.

TABLE 1
Chemical structures of nNOS inhibitors, in vitro NOS inhibition, and the
corresponding physicochemical data related to inhibition absorption and biomembrane
permeability

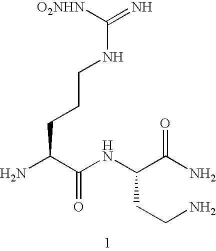

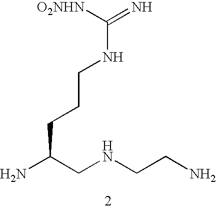

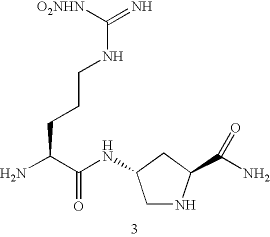

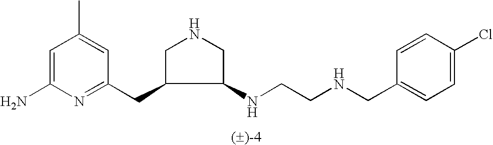

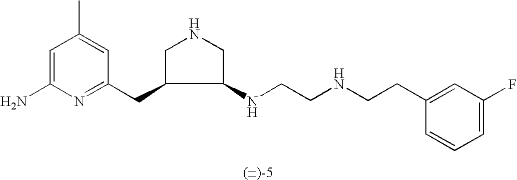

| | Kᵢ (μM) | | | Selectivity | | | | | | | |
|---|---|---|---|---|---|---|---|---|---|---|---|
| | nNOS | eNOS | iNOS | n/e | n/i | ClogP | LogD_{7.4} | TPSA^{a} | HBA^{b} | HBD^{c} | RB^{d} |
| εNNA | 0.57 | 0.75 | 4.55 | 1.3 | 8 | | | | | | |
| 1 | 0.13 | 200 | 25 | 1538 | 192 | −7.00 | −5.42 | 224.0 | 10 | 10 | 11 |
| 2 | 0.12 | 314 | 39 | 2577 | 320 | −5.79 | −5.88 | 163.8 | 7 | 8 | 10 |
| 3 | 0.10 | 128 | 29 | 1280 | 290 | −6.27 | −6.57 | 210.0 | 10 | 9 | 8 |
| (±)-4 | 0.085 | 85.2 | 9 | 1002 | 106 | 2.58 | −1.81 | 74.5 | 5 | 5 | 8 |
| (±)-5 | 0.014 | 28 | 4.1 | 2000 | 293 | 2.19 | −2.36 | 74.5 | 5 | 5 | 9 |

^{a}topological polar surface area (Å²);
^{b}Number of hydrogen bond acceptors;
^{c}Number of hydrogen bond donors;
^{d}rotatable bonds

Because the active site of nNOS is very polar and highly negatively charged, the pKₐ values of the fragments were considered in the inhibitor design (4 and 5). The 2-aminopyridine fragment was selected as the replacement for the guanidino group of the substrate L-Arg for binding to E592. A methyl group was introduced at the 4-position, mimicking the N^{ω}-alkyl-guanidino moiety in N^{ω}-methyl-L-Arg or N^{ω}-propyl-L-Arg, to improve contacts with a hydrophobic pocket opposite E592. Because the pKₐ values of 2-amino-6-methylpyridine and 2-amino-4,6-dimethylpyridine are 6.69 and 7.12, respectively (Paudler and Blewitt, 1966, J. Org. Chem. 31:1295-3717), these fragments act as a charge switch: for example, in the small intestine the fragment could be in its neutral form, which is favorable for absorption; in the NOS active site the local acidic environment (rat nNOS E592) converts it into the protonated (i.e., positively charged) form, which is favorable for binding.

The pyrrolidine fragment was chosen as the substitute for the α-amino group of 1-3 for two reasons. First, inhibitors 1-3, for example, adopt distinctly different conformations in eNOS and nNOS (Barker et al., 2006). By replacing the inhibitor α-amino group with a rigid pyrrolidine secondary amine, the pyrrolidine amino group is locked in a conformation for enhanced interactions with D597, favoring nNOS interaction. Second, a secondary amino group, for example, is more lipophilic and has less polar surface area, compared to a primary amino group, which is better, for example, for in vivo inhibitor delivery (Ertl et al., 2000, J. Med. Chem. 43:3714-3717).

In developing embodiments of the present invention, the ethylenediamine fragment was chosen, for example, to form electrostatic interactions and H-bonds with the heme propionate groups. Another reason to choose this fragment is the nitrogen atom in the pyrrolidine and the terminal nitrogen atom of the ethylenediamine fragment are expected to be higher pKₐ groups and, therefore, are protonated and positively charged; this would cause the nitrogen atom in the middle to have a low pKₐ and to be neutral, which is favorable, for example, for the inhibitor to permeate biomembranes.

Halogen-substituted phenyl fragments were introduced at the terminal amino group of the ethylenediamine fragment for three reasons: (1) The phenyl group can be stabilized in a very shallow hydrophobic pocket defined by M336, L337, Y706, and W306 (from the other subunit) (Flinspach et al., 2004, Biochem. 43:5181-5187). The residue that corresponds to rat nNOS L337 is N115 in murine iNOS or T121 in human iNOS. The hydrophobic aryl group is used to differentiate the hydrophobic L337 in nNOS from the polar N115 and T121 residues in murine and human iNOS, respectively. (2) The introduction of the phenylalkyl group renders the amino group of the ethylenediamine fragment a secondary amine and decreases its polar surface area, which is favorable for biomembrane permeability. (3) The introduction of halogen atoms at the para or meta positions of the phenyl group blocks/decreases the potential for metabolic degradation of the phenyl group as well as increases its lipophilicity.

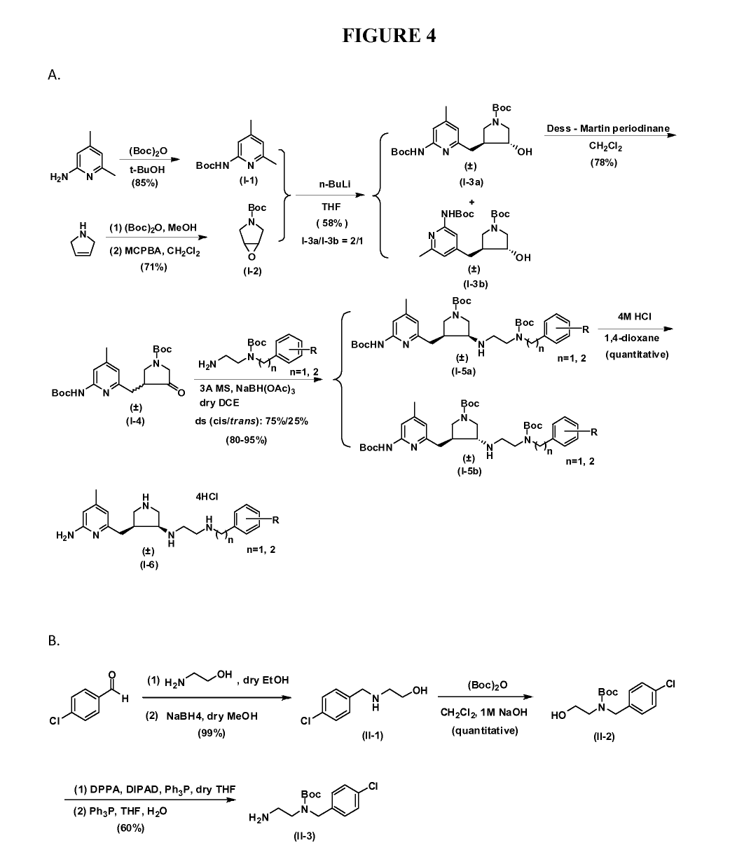

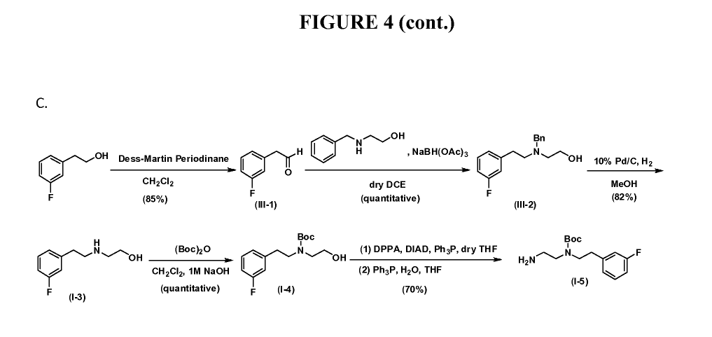

Exemplary synthetic routes for 4 and 5 are shown FIGS. 4A, B and C. Kinetic data and corresponding physical properties of 1-5 are shown in Table 1. When compared to 1-3, compounds 4 and 5 show higher inhibitory potency for nNOS. The selectivities of 4 and 5 for nNOS over eNOS and iNOS are similar to those of 1-3.

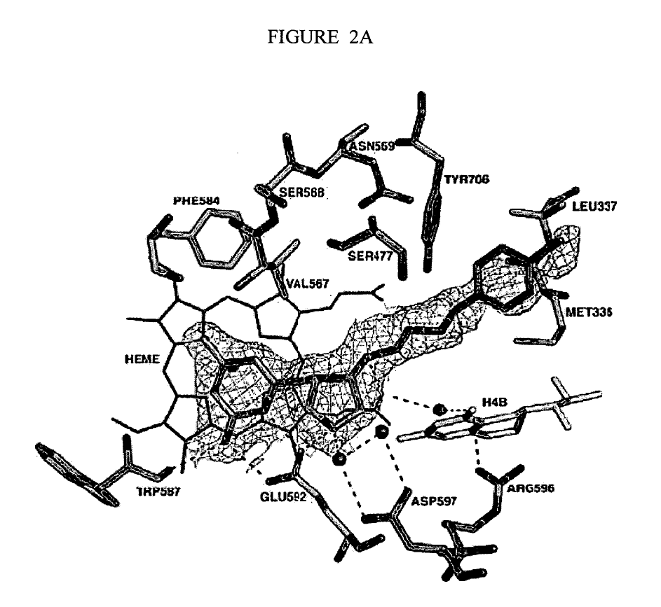

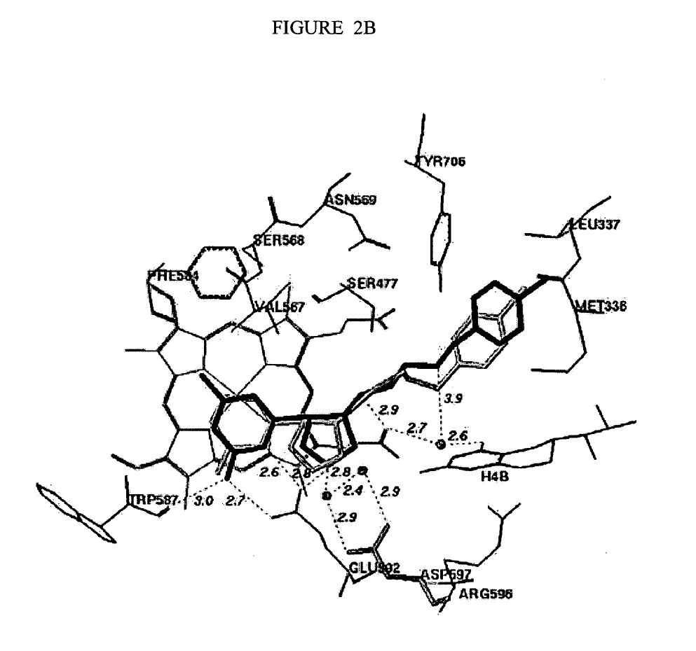

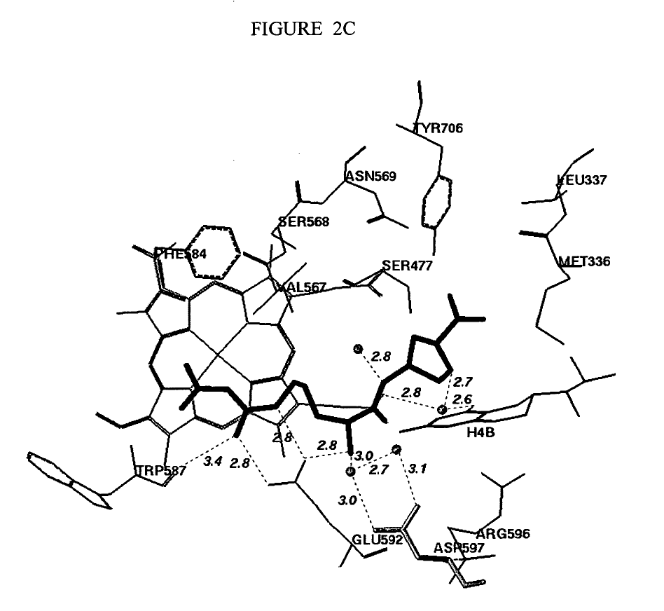

In developing embodiments of the present invention, the racemic mixture of 4 was used in cocrystallization with rat nNOS. Only one of the cis enantiomers, the (3′S,4′S)-isomer, was bound to the active site (FIG. 2A). The binding conformation of 4 in the crystal structure is very similar to that predicted in the fragment-based de novo design (FIG. 2B) and is very similar to the binding modes of 1-3 (FIG. 2C). The 2-aminopyridine group of 4 forms two H-bonds and electrostatic interactions with the carboxylic acid of E592, just as the nitroguanidino groups of 1-3 do. The nitrogen atom of the pyrrolidine ring of 4 forms a direct electrostatic interaction with E592 and is involved in a H-bond network with the carboxylic acid of D597 via two structural water molecules (FIG. 2B), just as the α-amino groups of 1-3 do (FIG. 2C). The NH group of the ethylenediamine fragment of 4 that is attached to the pyrrolidine ring forms a hydrogen bond with the heme propionate group, just as the amido NH groups of 1 and 3 and the secondary amino group of 2 do. The other nitrogen atom of the ethylenediamine fragment of 4 is involved in electrostatic interactions with heme propionate groups.

Although the previously-known fragment-based approaches could suggest a 2-aminopyridine fragment for binding with E592, it would be difficult to use those approaches to identify the pyrrolidine and ethylenediamine fragments, which are pivotal to nNOS selectivity and are not located in well-defined binding pockets (“hot spots”). The fragment hopping strategy described here, however, can easily identify these fragments, and the final structures (4 and 5) are more potent than, and of comparable selectivity to, 1-3. The predicted physico-chemical properties of 4 and 5, which are related to biomembrane permeability and absorption, are superior to those of the dipeptide amide compounds 1-3 (Table 1) and meet the basic virtual requirements for oral bioavailability and transport ability through the BBB. Thus, it is contemplated that 4 and 5 have better in vivo activities.

Because of the importance of eNOS to the regulation of cardiovascular activity, the effect of maternal administration of 4 and 5 on blood pressure in 22 day-gestation (E22) New Zealand white rabbit dams was determined. There was no effect on heart rate or on systolic or diastolic blood pressure by treatment with 4 or 5.

Next, the effect of maternal administration of 5 to E22 rabbit dams on the NOS activity and NOₓ levels in fetal brain was assessed to 1) show that 5 permeates the fetal brain, and 2) that 5 inhibits NOS activity in the fetal brain. The NOS activity assay in fetal brain homogenates assessed total constitutive NOS activity (i.e., both nNOS and eNOS). However, the in vitro studies indicated that eNOS is very poorly inhibited by compound 5. This also was demonstrated by the lack of in vivo effects on blood pressure. Therefore changes in NOS activity should correspond to changes in nNOS activity. Data showed that NOS inhibition was observed in fetal brain shortly after acute treatment with 5 was completed. The corresponding decrease in the NO concentration also indicated strong NOS inhibition had occurred.

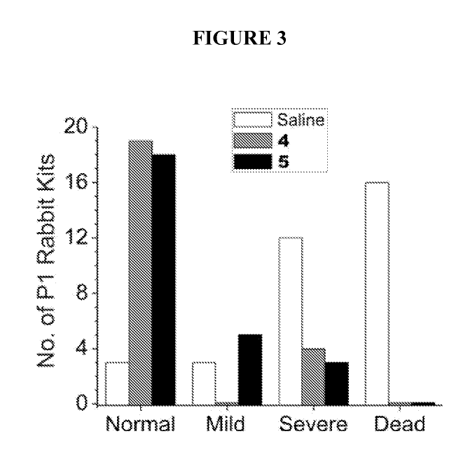

Next, experiments were conducted to determine whether compounds 4 and 5 could ameliorate the postnatal motor deficits suggestive of cerebral palsy. Saline, 4, or 5 were administered to E22 rabbit dams 30 minutes prior to and 30 min immediately after uterine ischemia for 40 min. The live newborn kits (postnatal day one; P1) were grouped into severe (postural deficits and/or hypertonia), mild (absence of hypertonia but presence of other abnormalities), or normal categories based on a standardized neurobehavioral examination. The results indicate a striking difference between the treated and untreated kits. The P1 kits born to saline-treated dams had a much greater incidence of discovered fetal/neonatal deaths and severe neurobehavioral abnormalities compared to those from dams treated with 4 or 5 (FIG. 3); in fact, there were no deaths observed with the 4- or 5-treated animals, which exhibited a substantially larger number of normal postnatal P1 kits (in two litters all 19 of the kits were normal). None of the compounds caused any detectable systemic toxicity in the rabbit dams. When administered post-ischemia, as expected in view of the results demonstrated herein, 5 exhibited no effect. This suggests that the nNOS activity already present at the beginning or produced during hypoxia-ischemia is crucial to the pathogenesis of motor deficits. NO present at the beginning of HI could result in formation of reactive nitrogen species, a pathway facilitated by superoxide produced by nNOS. These results also indicate that 4 and 5 are permeable to the fetal brain and are safe, powerful neuroprotective agents against HI-fetal brain injury, and the compounds are stable during the ischemic event. A comparison with 7-nitroindazole (7-NI), the standard for nNOS-selective inhibitor effects, showed that 4 and 5 are superior to 7-NI in their protection against HI-fetal brain injury (p<0.05 Fisher's Exact Test) and especially in comparison to their effects on the incidence of fetal death: none (0/49) was apparent with either 4 or 5, but 26% (10/38) of the fetuses of dams treated with 7-NI were stillborn. A need for more selective nNOS inhibitors has been reinforced by studies using another nNOS inhibitor, 1-(2-trifluoromethylphenyl) imidazole (TRIM), which shows a worse outcome following whole body ischemia.

In one embodiment, the present invention provides methods and compositions of preventing motor deficit disorders. In some embodiments, the present invention provides methods and compositions for preventing cerebral palsy. In some embodiments, the methods of prevention comprise administering a neuronal nitric oxide synthase inhibitor to a pregnant subject who is at risk of developing placental insufficiency such that oxygen flow to the fetus is compromised. In some embodiments, the methods of prevention can also be administered to subjects who face a birth such that potential asphyxia of the newborn to any degree is anticipated. In some embodiments, the methods of prevention can be applied to all pregnant subjects such that any risk to the fetus or newborn in developing cerebral palsy is decreased or eliminated.

In one embodiment, the present invention provides for compositions that are nNOS inhibitors and can be administered to pregnant subjects to prevent cerebral palsy. In some embodiments, the compositions comprise a compound as found in Formula I:

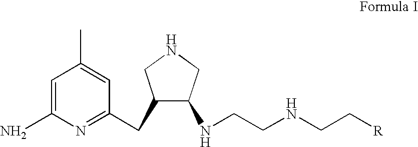

wherein R comprises a substituted or unsubstituted aryl group.

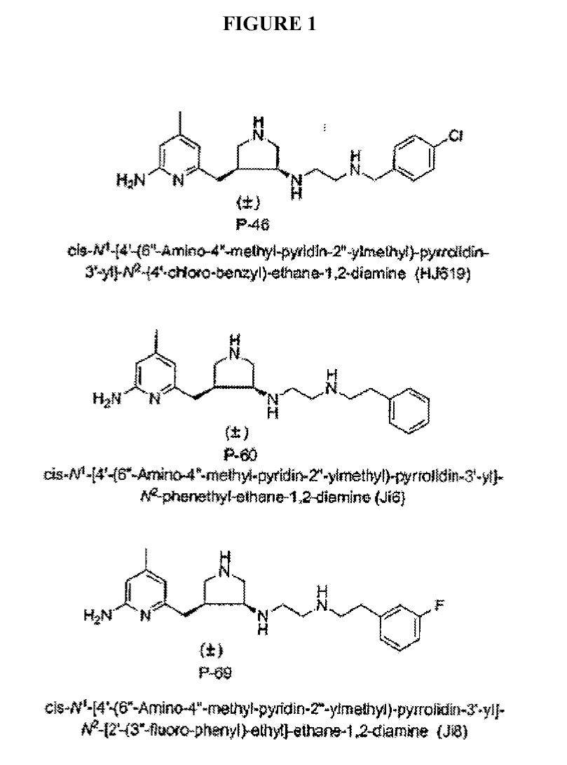

In some embodiments, the aryl group comprises R₁, where R₁ is hydrogen or a halogen (e.g., fluorine or chlorine). In some preferred embodiments, Formula I is the compound JI6 (FIG. 1) cis-N¹-[4′-6″-Amino-4″-methyl-pyridin-2″-ylmethyl)-pyrrolidin-3′-yl]-N²-phenylethyl-ethane-1,2-diamine.

In some embodiments, the compositions comprise a compound as found in Formula II:

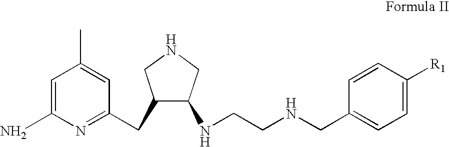

wherein R₁ is a halogen;
wherein R₁ is chlorine;
wherein R₁ is hydrogen.

In preferred embodiments, Formula II is compound HJ619 (FIG. 1) cis-N¹-[4′-6″-Amino-4″-methyl-pyridin-2″-ylmethyl)-pyrrolidin-3′-yl]-N²-(4′-chloro-benzyl)-ethane-1,2-diamine.

In some embodiments, the compositions comprise a compound as found in Formula III:

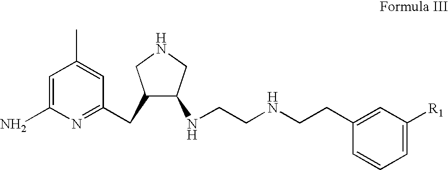

wherein R₁ is a halogen;
wherein R₁ is fluorine;
wherein R₁ is hydrogen.

In preferred embodiments, Formula III is compound JI8 (FIG. 1) cis-N¹-[4′-6″-Amino-4″-methyl-pyridin-2″-ylmethyl)-pyrrolidin-3′-yl]-N²-[2′-(3″fluoro-phenyl)ethyl]-ethane-1,2-diamine.

In some embodiments, the composition comprises any enantiomeric forms and racemic mixtures of any of the above compounds.

In one embodiment, the methods and compositions of the present invention are utilized with other preventative measures used to prevent cerebral palsy in fetuses or newborns. For example, administration of gamma globulin to Rh negative pregnant subjects to prevent mother/baby blood antigen incompatibilities, vaccinations of females to prevent viral infections, and the like.

In one embodiment, the compositions of the present invention are administered to the pregnant subject in a pharmaceutically acceptable solution, powder, elixir, etc. that is physiologically compatible with the pregnant subject. In some embodiments, the compositions are administered by injection, in other embodiments the compositions are administered orally. Those skilled in the art will recognize different avenues for medicament administration and what is required to render a medicament suitable for administration.

In one embodiment, the methods for preventing cerebral palsy in fetuses and newborns comprise administration of a nNOS inhibitor to a pregnant subject during pregnancy up until the baby has been delivered. In some embodiments, the nNOS inhibitor is administered directly to a fetus or to a newborn. In some embodiments, the administration is at least daily, at least every other day, at least weekly, at least bimonthly, at least monthly. Is preferred embodiments, a nNOS inhibitor that is administered to a subject is in a pharmaceutically acceptable form. For example, standard pharmaceutical formulation techniques can be employed, such as those described in Remington's Pharmaceutical Sciences, Mack Publishing Company, Easton, Pa. In some embodiments, a nNOS inhibitor is administered to a subject over a period of time (e.g., over 5 min., over 10 min., over 30 min., over 60 min., over a day, etc.). In some embodiments, the amount of a nNOS inhibitor administered is from 0.1-200 mg, from 1-200 mg, from 1-100 mg, from 2-125 mg, from 3-150 mg, from 4-175 mg, from 5-200 mg. In preferred embodiments, the amount of a nNOS inhibitor administered is from 5-100 mg. However, the invention is not limited to the particular dose ranges.

### EXAMPLES

### Example 1

### nNOS Inhibitor Treatment of Hypoxia-Ischemia of Rabbit Fetuses

In vivo global hypoxia-ischemia of fetuses was induced by uterine ischemia in timed pregnant New Zealand white rabbits (Myrtle's Rabbits, Thompson Station Tenn.) as previously described (Derrick et al., 2004, J. Neuro. 24:24-34; Tan et al., 2005, J. Clin. Neurol. 20:972-979). Briefly, the dams were anesthetized with intravenous fentanyl (75 ug/kg/hr) and droperidol (3.75 mg/kg/hr) and bag and mask ventilation provide to maintain normal arterial pH (7.35-7.45), pCO₂ (32-45 torr) and pO₂ (70-100 torr). Thereafter, the dams underwent spinal anesthesia by the administration of 0.75% bupivacaine through a 25 gauge spinal needle placed at L4-L5 intervertebral space. The fentanyl and droperidol dose was reduced by 60% to allow the dam to breathe spontaneously through a mask. Uterine ischemia that resulted in fetal hypoxia was induced with a 4F Fogarty arterial embolectomy catheter (Baxter Healthcare Corp., Santa Ana, Calif.). The catheter was introduced into the left femoral artery, advanced 10 cm into the descending aorta to above the uterine and below the renal arteries, and the balloon was inflated with 300 ul saline. Right lower extremity blood pressure was monitored by Doppler (Mini Dopplex D500, Huntleigh Technology, Luton UK) to ensure continued ischemia.

An intra-arterial injection of nNOS inhibitor (HJ619, JI6, or HJI8) was made 30 min prior to uterine ischemia and immediately after uterine ischemia. Deflating the balloon induced uterine reperfusion that resulted in fetal re-oxygenation. At the end of the procedure, the balloon was deflated and the catheter removed. The femoral artery was repaired with 7-0 sutures and the skin closed with 3-0 sutures. The mother was returned to her cage and allowed to deliver in a nest box, at term (31.5 days). On postnatal day one, the live born pups were subjected to a standardized set of behavioral tests and their tone assessed.

As seen in Table 2, the test pups whose mothers received placebo injection of saline mostly showed more perinatal deaths and severe motor dysfunction upon behavioral and tone analysis in the survivors, whereas the pups whose mothers received one of the three nNOS inhibitor injections demonstrated no deaths and less severe motor dysfunction in the survivors. The nNOS inhibitors tested include; JI8 (P-69) is cis-N¹-[4′-6″-Amino-4″-methyl-pyridin-2″-ylmethyl)-pyrrolidin-3′-yl]-N²-[2′-(3″fluoro-phenyl)-ethyl]-ethane-1,2-diamine, JI6 (P-60) is cis-N¹-[4′-6″-Amino-4″-methyl-pyridin-2″-ylmethyl)-pyrrolidin-3′-yl]-N²-phenylethyl-ethane-1,2-diamine and HJ619 (P-46) is cis-N¹-[4′-6″-Amino-4″-methyl-pyridin-2″-ylmethyl)-pyrrolidin-3′-yl]-N²-(4′-chloro-benzyl)-ethane-1,2-diamine.

TABLE 2

| Drug | Dose | Age | Normal | Moderate | Severe |
|---|---|---|---|---|---|
| Saline | Placebo | 22 | | | 7 |
| Saline | Placebo | 22 | | | 8 dead |
| JI 8 | 100 ki × 2 | 22 | 4 | 2 | |
| JI-8 | 100 ki × 2 | 22 | 5 | 2 | |
| JI-8 | 100 ki × 2 | 22 | | 1 | 3 |
| JI-8 | 100 ki × 2 | 22 | 9 | | |
| JI-6 | 200 ki × 2 | 22 | 9 | | |
| HJ619 | 75 ki × 2 | 22 | 9 | | 2 |
| HJ619 | 75 ki × 2 | 25 | | | 2 |
| HJ619 | 75 ki × 2 | 29 | 10 | | |

### Example 2

### Procedure for X-Ray Data Collection and Structure Refinement

The rat nNOS heme domain protein was generated and co-crystallized in the presence of inhibitor 4 according to procedures described in Kirk (2006, Curr. Top. Med. Chem. 6:1447-1456). Cryogenic (100K) X-ray data at 2.05 Å were collected at Advanced Light Source (Berkeley, Calif.), with 60556 unique reflections, 97.8% complete, and an overall R_{sym} of 0.052. The crystal belongs to space group P2₁2₁2₁ with cell dimensions a=52.21, b=111.53, c=164.84 Å. The binding of 4 was detected by difference Fourier technique using CNS. Model building and structure refinement were performed with O and CNS, respectively. The final model was refined to an R factor of 0.213 and a free R of 0.253 with good geometries. The coordinates and reflections of the structure were deposited to RCSB protein data bank with entry code 2O0N.

All publications and patents mentioned in the present application are herein incorporated by reference. Various modification and variation of the described methods and compositions of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A method of preventing a motor deficit disorder in a fetus or newborn infant by administering to a pregnant subject, or to the fetus or newborn, a neuronal nitric oxide synthase inhibitor.

2. The method of claim 1, wherein said nitric oxide synthase inhibitor is provided in a pharmaceutical formulation comprising a carrier.

3. The method of claim 1, wherein said nitric oxide synthase inhibitor comprises cis-N¹-[4′-6″-Amino-4″-methyl-pyridin-2″ylmethyl)-pyrrolidin-3′-yl]-N²-(4′-chloro-benzyl)-ethane-1,2-diamine.

4. The method of claim 1, wherein said nitric oxide synthase inhibitor comprises cis-N¹-[4′-6″Amino-4″-methyl-pyridin-2″-ylmethyl)-pyrrolidin-3′-yl]-N²-phenylethyl-ethane-1,2 diamine.

5. The method of claim 1, wherein said nitric oxide synthase inhibitor comprises cis-N¹-[4′-6″-Amino-4″-methyl-pyridin-2″-ylmethyl)-pyrrolidin-3′-yl]-N²-[2′-(3″fluoro-phenyl)-ethyl]-ethane-1,2-diamine.

6. The method of claim 1, wherein said nitric oxide synthase inhibitor comprises the structure of Formula I: 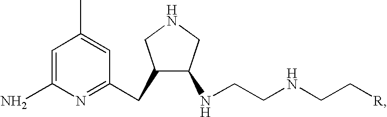
wherein R comprises a substituted or unsubstituted aryl group.

7. The method of claim 6, wherein the R is a substituted aryl group.

8. The method of claim 1, wherein said nitric oxide synthase inhibitor comprises the structure of Formula II: 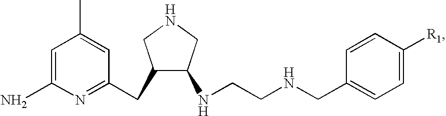
wherein R₁ is hydrogen or a halogen.

9. The method of claim 8, wherein the R₁ is chlorine.

10. The method of claim 1, wherein said nitric oxide synthase inhibitor comprises the structure of Formula III: 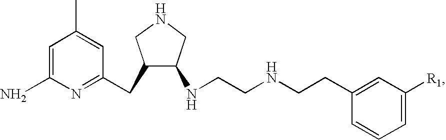
wherein R₁ is hydrogen or a halogen.

11. The method of claim 10, wherein the R₁ is fluorine.

12. The method of claim 1, wherein said motor deficit disorder is cerebral palsy.

13. The method of claim 1, wherein the pregnant subject is a subject is a subject identified as having a risk of delivering a fetus or newborn infant with a motor deficit disorder.

14. The method of claim 1, wherein said nitric oxide synthase inhibitor is administered to said pregnant subject.

15. The method of claim 1, wherein said nitric oxide synthase inhibitor is administered to said fetus or newborn.

16. The method of claim 1, wherein said pregnant subject is human.

17. The method of claim 1, wherein said fetus or newborn is a human.

18. Use of a neuronal nitric oxide synthase inhibitor for the manufacture of a medicament for use in the treatment of a fetus or newborn, by administration to a pregnant subject or to a fetus or newborn, of a motor deficit disorder.

19. A pharmaceutical composition comprising a neuronal nitric oxide synthase inhibitor and a second compound, said second compound comprising an agent useful for treatment of a motor deficit disorder that is not a nitric oxide synthase inhibitor.
